(19) United States
(12) Patent Application Publication
(10) Pub. No.: US 2004/0052843 A1
(43) Pub. Date: Mar. 18, 2004

(54) Controlled release dosage forms

(76) Inventors: E. Itzhak Lerner, Petach Tikva (IL); Vered Rosenberger, Givat Masuah Jerusalem (IL); Ofer Aqua, Ofra (IL); Moshe Flashner-Barak, Petach Tikva (IL)

Correspondence Address: KENYON & KENYON, ONE BROADWAY, NEW YORK, NY, 10004, US

(21) Appl. No.: 10/379,338

(22) Filed: Mar. 3, 2003

Related U.S. Application Data
(63) Continuation-in-part of application No. 10/291,619, filed on Nov. 12, 2002, now abandoned.
(60) Provisional application No. 60/342,442, filed on Dec. 24, 2001.
(60) Provisional application No. 60/361,821, filed on Mar. 4, 2002.

(51) Int. Cl. A61K 31/716; A61K 31/66; A61K 31/198; A61K 31/405; A61K 31/24

(52) U.S. Cl. 424/470; 514/102; 514/567; 514/419; 514/57; 514/540

(57) ABSTRACT

A zero-order release pharmaceutical dosage form for oral administration to a patient comprising a core tablet sheathed in an annular body of compressed powder or granular material is provided. A preferred embodiment of the zero-order release pharmaceutical dosage form is a solid pharmaceutical dosage form which reduces contact of the active ingredient in solid form with the mucosa lining the gastrointestinal tract, which is particularly advantageous for delivering an ulcerative drug. A process for making the zero-order release pharmaceutical dosage form are also provided.

### CROSS-REFERENCE TO RELATED APPLICATIONS

This application is a continuation-in-part application of U.S. application Ser. No. 10/291619, filed on Nov. 12, 2002 and claims the benefit of provisional application Serial No. 60/342,442, filed Dec. 24, 2001, and provisional application Serial No. 60/361,821, filed Mar. 4, 2002, both of which are incorporated herein by reference.

### FIELD OF THE INVENTION

The present invention relates to oral pharmaceutical dosage forms and more particularly to controlled release forms and forms designed to mask the taste of the active ingredient.

### BACKGROUND OF THE INVENTION

Tailoring drug delivery to the needs of therapy is a current goal in the development of drug delivery systems. The delivery profile may be desired to be one of immediate release within the oral cavity (the so-called “immediate dissolve” or “fast dissolve” systems), immediate release in the stomach or in the intestine, controlled slow release of the drug in the gastrointestinal (GI) tract, concomitant release of more than one drug at the same or at different rates, and many combinations of the above. There are systems that exist to provide drug delivery profiles that approximate the above requirements, but in each category there is room for improvement.

Immediate dissolve systems for immediate delivery of drugs in the oral cavity have been developed by R. P. Scherer Corporation in the form of a freeze dried tablet that readily dissolves on the tongue called Zydis® and by Cima labs, Inc. in the form of the OraSolv® system. These systems dissolve quickly in the mouth and are useful for cases where the delivery of the drug is needed immediately and in cases where the patient has difficulty swallowing tablets. Both of these systems suffer from being relatively fragile and very sensitive to moisture. They are therefore difficult to handle with the moisture of the fingers damaging the integrity of the delivery system (“melts on the hands and not in the mouth” to paraphrase an old advertisement).

In the world of controlled release drug delivery systems there have been certain axioms upon which much development has been based. One such axiom is that ‘flatter is better’ i.e. the flatter the delivery curve is vs. time the better the system will behave. It is therefore considered desirable to have delivery systems that give essentially a zero order release profile. The amount of drug released is not dependent on the amount left within the delivery system and remains constant over the entire delivery profile. Tailoring the drug delivery to the needs of the therapy is another axiom of delivery improvement. One can conceive of therapies that need a sudden burst of drug after several hours of constant delivery or a change in the rate of drug delivery after several hours.

A swelling hydrogel tablet delivery system or an eroding tablet delivery system, gives drug delivery that tapers off with time. In the eroding system, the surface that provides drug delivery is shrinking with time so the rate falls off proportionally. If the drug is delivered by diffusion through a non eroding hydrogel the rate falls off as drug depletion changes the force of the chemical gradient. These systems do not offer the opportunity to carefully tailor the drug release rates.

Zero order delivery has been achieved with the “Oros” osmotic pumps as is documented in many patents held by the Alza company (e.g. U.S. Pat. No. 3,995,631 to Higuchi, T. et. al., U.S. Pat. No. 3,977,404 to Theeuwes, F. and many other patents). The “Oros” system is based on osmotic pressure pushing the drug out of an almost microscopic orifice. The zero order profile is achieved due to the constant, small, cross section of the orifice being the rate determining step in the drug release. The “Oros” system has proven itself in several products but has limitations. It is most useful for soluble drugs with insoluble drugs having limited applicability. The technology of manufacture is somewhat complicated with the need of a laser drilled hole in the semipermeable coating. The drug release through an almost microscopic hole can also lead to several drawbacks. Clogging of the hole may limit drug release and the streaming of a concentrated solution of drug from the delivery system to the intestinal lumen can cause damage to the intestinal wall (see Laidler, P.; Maslin, S. C.; and Gihome, R. W. Pathol Res Pract 1985 180 (1) 74-76). Delays of the start of drug release can be achieved by coating the system (such as with an enteric coating) but the small orifice may be clogged by the coating and give erratic results in opening (if at all). The “Oros” system is best suited for a simple zero order delivery profile. Complicated patterns can be achieved with the “Oros” such as described in U.S. Pat. No. 5,156,850 to Wong, P. S. et. al. and in PCT WO 9823263 to Hamel, L. G. et. al. with concomitant complication of the manufacture and of the system, and without solving the drawbacks of the almost microscopic hole.

Zero order delivery profiles have been achieved with clever manipulation of the geometric surface of drug delivery as embodied in the “Geomatrix” delivery systems. (U.S. Pat. Nos. 4,839,177 to Colombo, P. et. al. and 5,422,123 to Conte, U. et. al. and assigned to Jagotech A G and many other patents). These systems achieve a zero order profile by sandwiching the drug delivery layer between two layers that are impermeable. Only the drug delivery layer is eroded and the cross-section of the eroding layer is constant. Again here, there are several drawbacks. The manufacture of the system requires special equipment to produce two and three layer tablets. The system does not easily lend itself to changing the rate of delivery during the release profile. The amount of drug available in the tablet is somewhat limited since only one of the layers is used for drug delivery. The zero order profile may not be followed up to 100% of drug release due to tablet breakup once most of the central layer has eroded.

In view of the foregoing, it would be highly desirable to have a versatile solid dosage form that enables controlled release of an active ingredient approaching zero order release. Accordingly, one object of the present invention is to provide a solid dosage form that can release a drug according to a predetermined release profile.

### SUMMARY OF THE INVENTION

The present invention provides controlled release pharmaceutical dosage forms in which a core tablet is sheathed in an annular body of compressed powder or granular material.

The drug layer may be recessed from the opening of the annular body on one or both sides. The drug layer is recessed from the surface so that any contact, whether with hands or with the mucosa, is with the walls of the annular body. The annular body is preferably made of non ulcerative and non sensitive pharmaceutical ingredients such as hydroxypropyl cellulose, hydroxypropyl methylcellulose, microcrystalline cellulose, starch, lactose, sugars, polyvinyl pyrrolidone, calcium phosphate and any other regular tablet excipients.

The controlled release pharmaceutical dosage forms of the invention release the active ingredient from the core tablet into the environment of the dosage form at a rate in the range of from 3% per hour to 12% per hour.

The present invention further provides a pharmaceutical dosage form wherein the pharmaceutical dosage form is adapted for extended or zero-order release of active drug material.

The present invention further provides a pharmaceutical dosage form wherein the pharmaceutical dosage form is adapted for immediate release of active drug material.

The present invention further provides a pharmaceutical dosage form wherein the pharmaceutical dosage form is adapted for sublingual administration.

The present invention further provides a pharmaceutical dosage form wherein the pharmaceutical dosage form is adapted so as to mask the taste of the active material.

The present invention further provides a method of independently controlling the rate of release of coactive ingredients in a single dosage form.

The present invention further provides a pharmaceutical dosage form for co-administration of coactive ingredients in a single dosage form.

### BRIEF DESCRIPTION OF THE FIGURES

FIG. 1 shows sectional perspective, side and top down views of a solid dosage form with a recessed core tablet of active ingredient in a compressed annular body of powder or granular material in accordance with the invention.

FIG. 2 is a perspective view of a single station tableting press shown with the toolset installed.

FIG. 3 is a sectional side view of the columnar punch and punch assembly.

FIGS. 4a-4e are sectional side views depicting stages in a cycle of operation from delivery of powder or granular material to ejection of a finished tablet at a tableting station equipped with a toolset in accordance with the invention.

FIG. 5 is a plot of the average rate of alendronate excretion in urine of humans who had taken a dosage form in accordance with the present invention containing 70 mg monosodium alendronate and a prior art 70 mg monosodium alendronate dosage form.

FIG. 6 is a plot of the rate of release of oxybutynin from a dosage form in accordance with the invention, wherein the rate of release is maintained between 3% h⁻¹ and 12% h⁻¹ for seven hours or more.

FIG. 7 is a plot of the rate of release of oxybutynin from a dosage form in accordance with the invention. The proportion of hydrogel in the core tablet is increased relative to the dosage form that produced FIG. 6 resulting in a decreased maximum rate of release and an extended release between 3% and 12% per hour for about twelve hours.

FIG. 8 is a plot of the rate of release of oxybutynin from a dosage form in accordance with the invention. The proportion of release-inhibiting hydrogel in the annular body was increased relative to the dosage form that produced FIG. 7. The maximum rate of release was further reduced to less than 7% h⁻¹.

FIG. 9 is a plot of the rate of release of carbidopa from the core tablet and of levodopa from the annular body of a dosage form in accordance with the present invention. The core tablet is cylindrically shaped and annular having a 2.5 mm diameter hole therethrough.

FIG. 10 is a plot of the rate of release of carbidopa from the core tablet and of levodopa from the annular body of a dosage form in accordance with the present invention. The core tablet of this dosage form has a 4.6 mm hole, larger than that in the dosage form that produced FIG. 9, resulting in greater surface area and a more rapid rate of release of carbidopa.

FIG. 11 is a plot of the rate of release of carbidopa from the core tablet and of levodopa from the annular body of a dosage form in accordance with the present invention. The dosage form that produced this figure had an oval core tablet with a 3 mm hole therethrough which resulted in a release similar to the cylindrical core table with a 2.5 mm hole (FIG. 9).

### DESCRIPTION OF THE PREFERRED EMBODIMENTS

The present invention provides a novel solid dosage form, as well as tooling and a process for producing the novel dosage form. Preferred embodiments of the invention are well suited for the controlled release of drugs, especially extended release approaching zero-order, and for taste masking of unpleasant tasting drugs.

The novel dosage form comprises a core tablet containing an active pharmaceutical ingredient sheathed in an annular body (also called a mantle in this disclosure) comprised of compressed powder or granular material. The core tablet has first and second opposed surfaces and a circumferential surface. “Sheathed” means that the annular body encircles the core tablet and is in contact with the core tablet about its circumferential surface, but leaves opposed surfaces of the core tablet substantially exposed. The core tablet contains at least one active pharmaceutical ingredient, but otherwise its formulation is not critical to the invention. The core tablet can be formulated for any desired release profile, such as immediate release, delayed release, burst or pulsed release, sustained or zero order release. The annular body can be formulated to achieve any desired purpose, such as gastric retention, ease of swallowing, taste masking and control of the rate of drug release from the core tablet. The annular body also can contain or be coated with a co-active ingredient.

The terms “drug” and “active pharmaceutical ingredient” broadly include any biologically, physiologically, or pharmacologically active the agent. Active pharmaceutical ingredients that can be administered in the compressed dosage form of the present invention include adrenergic receptor agonists and antagonists; muscanic receptor agonists and antagonists; anticholinesterase agents; neuromuscular blocking agents; ganglionic blocking and stimulating agents; sympathomimetic drugs; serotonin receptor agonists and antagonists; central nervous system active drugs such as psychotropic drugs, antipsychotic drugs, antianxiety drugs, antidepressents, antimanic drugs, anesthetics, hypnotics, sedatives, hallucinogenic drugs and antihallucinogenic drugs; antiepileptic drugs; antimigraine drugs; drugs for treatment of Parkinson's, Alzheimer's and Huntington's disease; analgesics; antitussive agents; antihistaminic drugs; H₁, H₂, and H₃ receptor antagonists; bradykinin receptor antagonists; antipyretic agents; antiinflammatory agents; NSAIDs; diuretics; inhibitors of Na⁺—Cl⁻ symport; vasopressin receptor agonists and antagonists; ACE inhibitors; angiotensin II receptor antagonists; renin inhibitors; calcium channel blockers; β-adrenergic receptor antagonists; antiplatelet agents; antithrombic agents; antihypertensive agents; vasodilators; phosphodiesterase inhibitors; antiarrhythmic drugs; HMG CoA reductase inhibitors; H⁺, K⁺-ATPase inhibitors; prostaglandins and prostaglandin analogs; laxatives; antidiarrheal agents; antiemetic agents; prokinetic agents; antiparasitic agents such as antimalarial agents, antibacterial agents, drugs for treatment of protozoal infections and antihelmintic drugs; antimicrobial drugs such as sulfonamides, quinolones, β-lactam antibiotics, aminoglycosides, tetracyclines, chloramphenicol and erythromycin; drugs for treatment of tuberculosis, drugs for treatment of leprosy; antifungal agents; antiviral agents; antineoplastic agents; immunomodulators; hematopoietic agents; growth factors; vitamins; minerals; anticoagulants; hormones and hormone antagonists such as antithyroid drugs, estrogens, progestins, androgens, adrenocortical steroids and adrenocortical steroid inhibitors; insulin; hypoglycemic agents; calcium resorption inhibitors; glucocorticoids; retinoids and heavy-metal antagonists.

The annular body can be formed of any powdered or granular pharmaceutically acceptable excipients and can itself include a pharmaceutically active ingredient. In particular, it may be mentioned that diluents, binders, disintegrants, glidants, lubricants, flavorants, colorants and the like can be included in the annular body. Powdering and granulation with conventional excipients and the techniques for forming compressed bodies therefrom with given characteristics in terms of friability, hardness and freedom from capping is well within the knowledge of those skilled in the art of tableting.

Preferred excipients for forming the annular body include hydroxypropyl cellulose (e.g., Klucel™), hydroxypropyl methylcellulose (e.g. Methocel™), microcrystalline cellulose (e.g., Avicel™), starch, lactose, sugars, polyvinylpyrrolidone (e.g., Kollidon™, Plasdone™) and calcium phosphate.

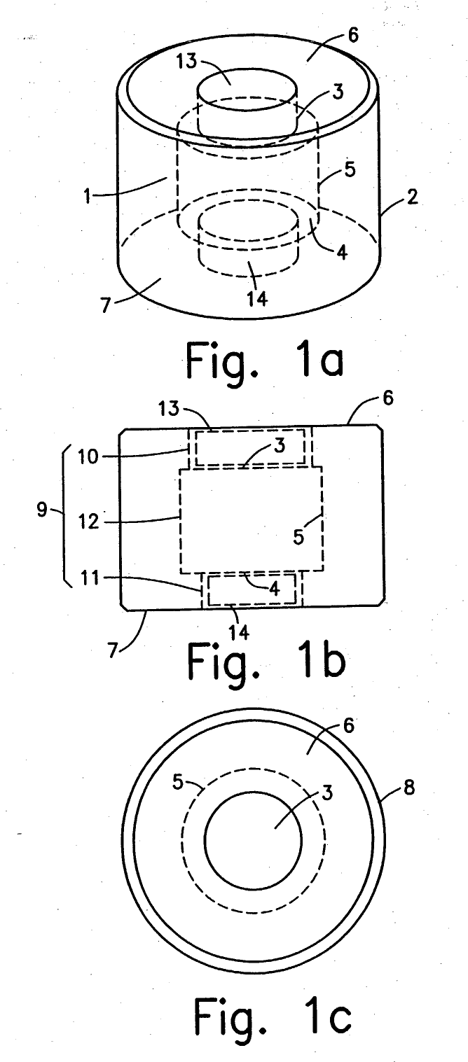

In an especially preferred compressed dosage form illustrated in FIG. 1, core tablet 1 containing the active pharmaceutical ingredient is recessed in the annular body 2, which is composed of non-ulcerative pharmaceutical excipients. The “recessed” tablet is especially well suited for oral delivery of ulcerative drugs. It reduces the incidence of pill esophagitis and contact gastritis by localizing the ulcerative drug in a core tablet that is shielded from contact with the mucosa lining the gastrointestinal tract. The drug is shielded because the core tablet is recessed. Recessing the core tablet does not significantly alter the release profile of the core tablet because a sizable portion of the surface of the core tablet is in fluid communication with the environment. In contrast, in coated or encapsulated dosage forms, the coating or capsule must be breached by gastric fluid before the drug is released. In the present invention, the outer contour of the dosage form protects the mucosa lining the gastrointestinal tract without interrupting fluid communication between the core tablet and the environment.

Exemplary of drugs that can be advantageously delivered using the preferred recessed dosage form of this invention are monosodium alendronate monohydrate, monosodium alendronate trihydrate, sodium etidronate, sodium risedronate, pamidronate, aspirin, ibuprofen, naproxen, fenoprofen, ketoprofen, oxaprozin, flubiprofen, indomethacin, sulindac, etodolac, mefenamic acid, meclofenamate sodium, tolmetin, ketorolac, diclofenac, piroxicam, meloxicam, tenoxicam, phenylbutazone, oxyphenbutazone, oxybutynin, alendronate, carbidopa, levodopa, tizanidine, sumatriptan, pharmaceutically acceptable salts, hydrates, isomers, esters and ethers thereof, and mixtures thereof.

Both the core tablet and the annular body may be formed into any suitable shape. Specific shapes can be achieved by use of specifically designed punches. Preferably the core tablet and the annular body are cylindrical in shape. The core tablet and the annular body may be the same or different in shape. The exposed surfaces of the core tablet may be of any suitable shape. Preferably, the exposed surfaces of the core tablet are circular or oval.

Turning again to FIG. 1, core tablet 1 has opposed first and second surfaces 3 and 4 and an outer circumferential surface 5 extending between the opposed surfaces. Core tablet 1 is preferably cylindrical or disk shaped for ease of manufacture, but need not be so. In a dosage form for administration to humans, the maximum distance across either of the opposed surfaces 3 or 4 is preferably from about 2 mm to about 12 mm, more preferably from about 4 mm to about 7 mm, most preferably about 5 mm. Opposed surfaces 3 and 4 can be flat, concave or convex and are preferably flat for bearing modest axial compression forces exerted by flat pressing surfaces during formation of the annular body about the core tablet.

In outer contour, annular body 2 is preferably cylindrically shaped, but it can have any cross section, such as oval, elliptical or oblong. The outer diameter is preferably of from about 5 mm to about 15 mm, more preferably of from about 7 mm to about 12 mm, most preferably about 9 mm. The inner diameter can be any size up to about 2 mm less than the outer diameter. A narrow inner diameter less than 2 mm may slow release of the drug if an excipient in the annular body swells upon contact with gastric fluid. However, in some embodiments, a lower limit 0.5 mm may still be useful. Preferably, the inner diameter is 3 mm or greater.

Annular body 2 has opposed first and second annular faces 6 and 7, an outer circumferential surface 8 extending between the annular faces from their outer edges, and an inner circumferential surface 9 extending between the annular surfaces from their inner edges, thus defining an annulus.

As best seen in side view (FIG. 1B), inner circumferential surface 9 of annular body 2 consists of three longitudinal (axial) segments. First and second segments 10 and 11 are terminal and do not contact the sides of the core tablet. They are separated by an internal third segment 12 that contacts the outer circumferential surface 5 of core tablet 1. Opposed surfaces 3 and 4 of the core tablet are therefore recessed from annular faces 6 and 7 of the annular body. Opposed surfaces 3 and 4 are preferably recessed from about 0.5 mm to about 4 mm, more preferably about 1.5 mm relative to the annular faces 6 and 7 of the annular body (said recessed distance corresponding to the length of the corresponding terminal segment). The recess depth of surfaces 3 and 4 can be the same or it can be different.

By recessing the drug-containing core tablet, any contact between the dosage form and the gastrointestinal mucosa occurs with a surface of the annular body formed of non-ulcerative excipients, and optionally one or more non-ulcerative co-active ingredient, rather than with the solid ulcerative active ingredient. However, one or both of opposed surfaces 3 and 4 can be flush with annular faces 6 and 7 of the annular body without deleterious effect when the dosage form of the present invention is used to administer non-ulcerative drugs.

To better apprehend the preferred recessed dosage form embodiment of the invention, it is useful to conceive of surface 3 of the core tablet and first longitudinal segment 10 as defining a first void 13. Likewise, surface 4 of the core tablet and second longitudinal segment 11 define a second void 14. Voids 13 and 14 fill with gastric fluid when the dosage form is immersed in gastric fluid after reaching the stomach. Gastric fluid passes through the voids to contact the core tablet and the drug leaves through the voids after it is dissolved. Voids 13 and 14 are preferably from about 0.5 mm to about 10 mm, more preferably from about 3 mm to about 6 mm and most preferably about 4.5 mm in width (measured parallel to first or second opposed surfaces). Drug release, therefore, does not occur by an osmotic mechanism such as occurs with pierced dosage forms made using the apparatus of U.S. Pat. No. 5,071,607. Rather, in a large still fluid environment, drug concentration drops off roughly isotropically and exponentially by diffusion. In contrast, osmotic release of the drug product would produce a streaming flow that can cause locally high concentrations of the drug and osmotic agents at considerable distance from the tablet. Osmotic streams highly concentrated in an ulcerative drug are potentially irritating to the mucosa, just like the solid drug, particularly if the tablet is lodged in a fold in the gastrointestinal wall.

Opposed surfaces 3 and 4 of the core tablet are preferably substantially exposed, i.e. are not substantially covered by the annular body. “Substantially exposed” means that less than about 50% of each of the opposed surfaces is concealed or hidden from visual inspection by the annular body. A portion of opposed surfaces 3 and 4 can be concealed by the annular body because of differences between the diameter and shape of the core tablet and the diameter and shape of certain pressing portions of the tooling used to compress the annular body, as will become apparent from consideration of the description of the tooling aspect of the invention. Such differences may result in inner segment 12 being offset from terminal segments 10 and 11, which, themselves, can have different longitudinal cross sections, e.g. have different diameters, as depicted in FIG. 1. Alternatively, the cross section of the annulus defined by inner circumferential surface 9 can be uniform throughout its length. Although a portion of opposed surfaces 3 and 4 can be concealed by the annular body that is not necessarily the case.

Further, the invention contemplates that the rate of release of the drug is determined by the formulation and shape of the core tablet, not by diffusion of the drug through the annular body which contributes to the versatility of the dosage form for different release profiles.

In one embodiment, the pharmaceutical dosage form is an extended release dosage form. Active drug material is delivered via the exposed axial surfaces of the core tablet. The exposed axial surfaces retain a constant cross-section during delivery of the active material, thus producing a zero-order release profile. For extended release applications, the core tablet can be formulated to be of an eroding or diffusive nature.

An extended release core tablet preferably contains a hydrogel such as hydroxypropyl methylcellulose, hydroxypropyl cellulose, ethylcellulose and the like. Optionally, the core tablet also contains a more rapidly dissolving substance like compressible sucrose to open pores in the hydrogel matrix and thereby modulate the hydrogel grip on the active ingredient. In a zero order extended release dosage form wherein the active ingredient is contained in the core tablet, the annular body will be formulated to be yet slower dissolving than the core tablet so that the surface area of the core tablet will remain constant. Mixtures of about 1 part high molecular weight polyethylene glycol (PEG) and 3-5 parts ethyl cellulose will retain their shape and rigidity in water for the time that it takes for most conventional eroding or swelling hydrogel matrices to completely release the drug. An especially preferred composition of the annular body of an extended release dosage form in accordance with this invention comprises about 15-25 parts PEG 4000, about 70-80 parts ethylcellulose and about 5 parts polyvinylpyrrolidone. The rate of release of active material from the core tablet of extended release dosage forms is less than about 15% by weight per hour. Preferably the rate of release is from about 3% per hour to about 12% by weight per hour. Extended release dosage forms are adapted for the release of active material over a period of at least about 4 hours, more preferably at least about 7 hours, and most preferably at least about 10 hours. The rate of release of active ingredient is measured in a United States Pharmacopeia standard apparatus II solution tester in an aqueous solution buffered at 6.8 at 37° C. with a stirring rate of 50 revolutions per minute.

Dosage forms in accordance with this invention also are adaptable for immediate release and have unique advantages when used for immediate release. The annular body or sheathing layer provides protection for the immediate release core tablet while being handled by the patient or caregiver. The core drug layer is recessed from the surface so that any contact is with the walls of the annular body. While the core tablet may be fragile, ones hands would contact only the non fragile annular body. The core tablet can be formulated to be of a “fast dissolve” nature without the drawbacks of the current “fast dissolve” systems. The drug can be released by dissolution into the saliva as the “fast dissolve” form is held in the mouth for a few minutes. The outer annular body can be formulated to dissolve too but at a slower rate so that it not be as sensitive to moisture or alternately could be swallowed (by those that can swallow a tablet) or expectorated. Dissolution of the released drug is preferably carried out in less than about 5 minutes, more preferably in less than about 2 minutes. The rate of dissolution of active ingredient is measured in a United States Pharmacopeia standard apparatus II dissolution unit at 37° C. or a United States Pharmacopeia standard apparatus II dissolution unit at 37° C. with stirring at 50 revolutions per minute. The dosage form can be formulated so as to be suitable for rapid dissolution in the oral cavity without co-administration of liquid.

As a consequence of the protection afforded by the annular body, many active ingredients can be used in a greater proportion in the core tablet formulation than they could be in conventional tablets. Thus, a core tablet can contain a very high concentration of active drug material without thereby producing a dosage form that is too delicate to be handled. An immediate release core tablet preferably contains a superdistintegrant. Other preferred excipients for an immediate release formulation include sodium saccharin, microcrystalline cellulose, lactose and menthol.

One immediate release core tablet formulation that has been found to compress well in the tooling of the invention contains 5 parts active ingredient, 20 parts crospovidone, 74 parts MicrocelLac®, 1 part lubricant and 0.4 parts menthol.

When the core tablet is formulated for immediate release, the annular body can be formulated differently than the annular body of an extended release formulation because it does not need to remain rigid for as long a time. The annular body will generally be formulated to dissolve more slowly than the core tablet, however. As further illustrated in Example 3, an annular body can be made by modifying an immediate release core formulation by reducing the proportion of superdisintegrant, and optionally substituting a dissolving, but non-swelling excipient, like compressible sugar.

Immediate release dosage forms in accordance with the invention are useful for administering active pharmaceutical ingredients that have an unpleasant taste, like sumatriptan succinate. One method of achieving taste masking includes recessing the surface of the core tablet within the annular body, thus avoiding contact between the tongue and the core tablet. Immediate release dosage forms in accordance with the invention also are useful for sublingual and buccal administration of drugs. It is often desirable that a sublingually administered drug be released from the dosage form as rapidly as possible. Buccal administration can also be via immediate release dosage forms. To achieve rapid release, such dosage forms can be formulation with a high proportion of the active ingredient. However, a high proportion of active ingredient will, in many cases, make the tablet fragile. As previously discussed in another context, the annular body protects fragile core tablets in the dosage forms of this invention, making them well adapted for sublingual and buccal administration of drugs. Preferred drugs for sublingual and buccal administration in the dosage forms of the present invention are tizanidine, nitroglycerin, isosorbide dinitrate, isosorbide mononitrate, vaccines, ergotamine and other anti-migraine compounds, lorazepam and other tranquilizers, vitamin B12 and folic acid, and mixtures thereof. A dosage form of tizanidine is further illustrated in Example 3.

The rate of release of active material from the core tablet of immediate release or sublingual dosage forms is greater less than about 90% in 30 minutes. Preferably the rate of release is greater than about 85% in 15 minutes. The rate of release of active ingredient is measured in a United States Pharmacopeia standard apparatus II dissolution unit at 37° C. or a United States Pharmacopeia standard apparatus II dissolution unit at 37° C. with stirring at 50 revolutions per minute.

The core tablet also can be a bilayer tablet with each layer containing the same or different drugs and each layer releasing the drug at the same or at different rates. One of the layers could be an immediate release layer and the other a slow release layer, or both can be slow release layers. The inner tablet can be formulated to be a three layer tablet with the central layer being a drug to be delivered after a delay. The two outer layers can be delay layers or drug delivery layers with the same or different drugs and with the same or different release profiles. The middle layer can contain again the same or different drugs compared to the outer layers and can be of a controlled release or an immediate release nature. Thus, one can have controlled release of two drugs each at its desired release rate and a delayed release or delayed pulse of a third drug. The currently described invention thus gives a very wide range of drug delivery capabilities not addressed by conventional dosage forms and improves upon the performance of other known delivery systems.

Dosage forms in accordance with the invention also can be formulated to deliver two drugs by locating one of the drugs in the core tablet and the other in the annular body. Such an arrangement enables the release rate of each active ingredient to be controlled independently by formulation adjustments to the portion of the dosage form, i.e. core tablet or annular ring, that contains the drug that is being released either too slowly or too quickly. In addition, the shape of one of the portions can be changed without adjusting the formulation. For instance, the powder or granular material may be pressed around the core tablet into a body having an oval cross-section rather than a circular cross-section to achieve a faster rate of release (resulting from increased surface area). In addition, the core tablet may have a hole extending from one axial face to the other in order to increase the surface and thereby increase the release rate. The release rate can be further controlled through changes to the diameter of the hole, as further illustrated in Example 4.

Preferred drug combinations for use with the invention include levodopa/carbidopa, acetaminophen/caffeine, acetaminophen/codeine, acetaminophen/antihistamines, vitamin and mineral combinations and combinations of antibiotics. The combination of levodopa/carbidopa is especially preferred. In Example 5, especially preferred levodopa/carbidopa dosage forms are illustrated wherein the levodopa is dispersed in a hydrogel matrix in the annular body and carbidopa is direct compressed with a direct compression excipient mix and a superdistintegrant in the core tablet.

The rate of release of levodopa material from the core tablet of a combination levodopa/carbidopa drug dosage forms is less than about 35% by weight per hour. Preferably the rate of release is from about 3% per hour to about 30% by weight per hour, more preferably from about 6% per hour to about 30% per hour. Levodopa/carbidopa combination dosage forms are adapted for the release of active material over a period of at least about 2 hours, more preferably at least about 3 hours. The rate of release of active ingredient is measured in a United States Pharmacopeia standard apparatus II solution tester in 0.1N HCl at 37° C. with a stirring rate of 50 revolutions per minute.

The solid dosage forms with a drug-containing core tablet sheathed in a compressed annular body of non-ulcerative excipients can be produced using a novel toolset that constitutes a second aspect of the invention.

The toolset can be used in conjunction with conventional tablet presses such as rotary presses and reciprocating presses or with presses that have been specially designed and manufactured. Examples of commercially available rotary presses are the Manesty Express 25, the Kilian RUD or RTS series and comparable equipment. Examples of commercially available reciprocating presses are the Manesty F3 and comparable equipment made by Stokes, Kilian and Key Industries.

The principle elements of the toolset are a columnar punch and a punch assembly comprising an annular punch having an annulus (or bore), a core rod slidably engageable within the annulus of the annular punch, wherein the core rod is capable of movement between a retracted position and an extended position, the core rod being biased in the extended position. The columnar punch and punch assembly are sized and shaped to fit into the die bore of a rotary or reciprocating tablet machine.

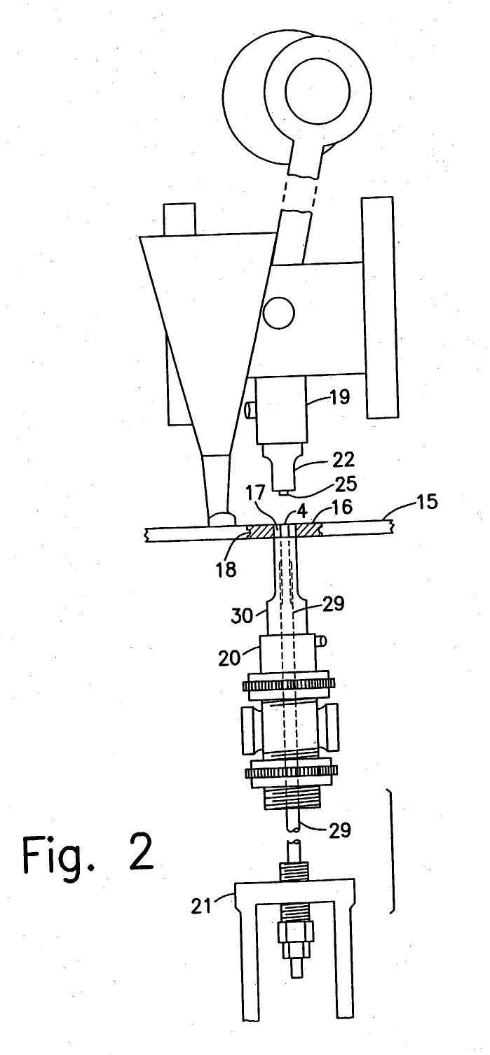

The toolset is well adapted for use with conventional single station tablet presses in which opposing upper and lower punches cooperatively compress a powder or granular material within a die. Referring to FIG. 2, single station presses are provided with a horizontal die table 15 having an aperture for receiving a die 16 and associated gripping means for locking the die into position. Dies for such presses customarily have opposed flat surfaces with a centrally located bore 17 having a highly polished wall surface extending from surface to surface and a circumferential locking groove 18 for engaging the gripping means. The bore serves as a receptacle for receiving powder or granular material to be compressed when the lower punch is partially inserted. The rims of the bore are customarily chamfered to help guide the punches into the bore. The bore cross section determines the size and shape of the finished tablet in cross section. The quantity of material and pressure of compression determine the tablet height. The bore can be cylindrical, but also can be any other shape.

In operation, the bore is filled with material and the upper punch is inserted into the bore and pressed against the material under high pressure thereby compressing the powder or granulated material into a tablet between the pressing, or contact, surfaces of the punches.

Together, the wall of the bore and the contact surfaces of the upper and lower punches define a mold that determines the size and surface contours of the final product. The final product can have any external contour by selection of appropriate bore shape and contact face contour.

After compression, the upper punch is withdrawn and the lower punch is advanced to eject the tablet.

The upper and lower punches are advanced and withdrawn by independently actuated upper and lower reciprocating rams 19 and 20. Customarily, single punch presses are also provided with a stationary mounting point 21 below the die table coaxial with the aperture.

A toolset of this invention adapted for use in a single station press comprises a columnar punch and a punch assembly comprising a collar, core rod and annular punch.

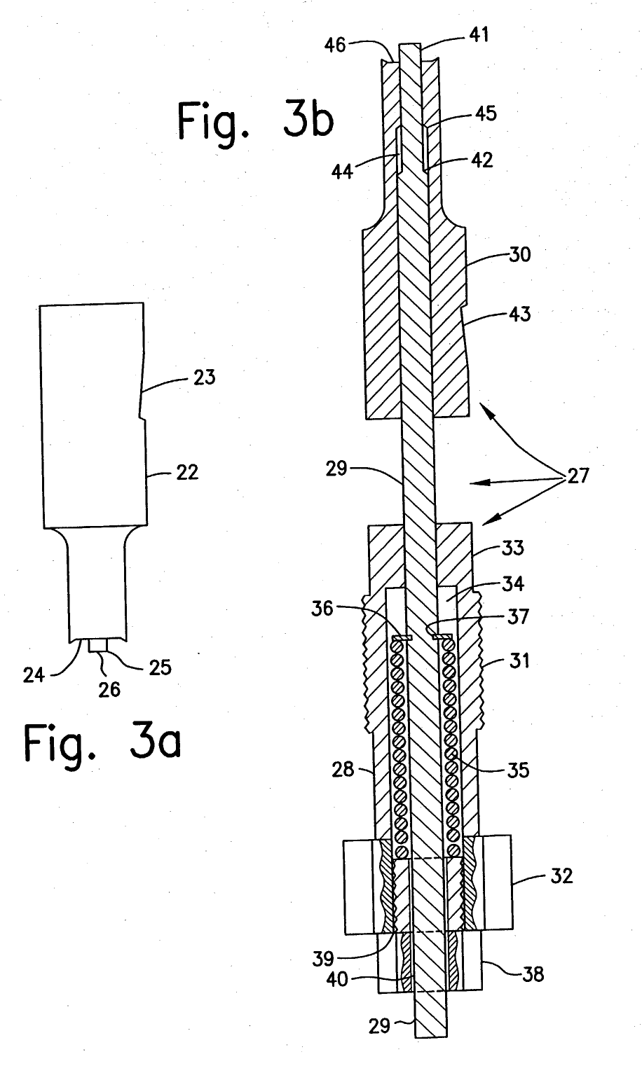

Referring now to FIG. 3, columnar punch 22 can be of a conventional columnar shape and is provided with locking means, such as locking flat 23 to secure it to the upper reciprocating ram 19 of the tablet press.

Columnar punch 22 includes a contact face 24. Contact face 24 can have any desired contour, e.g. standard concave, deep concave, extra deep concave, modified ball or flat. Preferably, the contour of contact face 24 is flat with a beveled edge.

A columnar punch for use in producing a dosage form of the present invention having a recessed core also has a protrusion 25 centrally located on the contact face 24, as illustrated. Preferably, the height of protrusion 25 is from about 0.5 mm to about 4 mm, more preferably about 1.5 mm. The shape of the protrusion is preferably cylindrical or tapered cylindrical but can also be oval, ellipsoid, oblong or any other shape desired. The protrusion is preferably cylindrical and has a flat raised surface 26. Protrusion 25 preferably has a diameter of from about 3 mm to about 7 mm, more preferably about 4.5 mm. In other embodiments, particularly suited to use when non-ulcerative active pharmaceutical ingredients are to be administered, protrusion 25 is absent.

Punch assembly 27 comprises collar 28, core rod 29 slidably engaged with collar 28 and annular punch 30 slidably engageable with core rod 29.

Collar 28 is provided with mounting means, such as external threads 31 around its circumference for mounting to stationary mounting point 21 located below the die table. As illustrated, the distal end 32 of collar 28 relative to the die table when installed, has a gripping section (shown with optional hexagonal cross section) for gripping by a wrench for mounting to stationary mounting point 21. At the proximal end 33 of the collar 28 relative to the die table when installed, the annulus is dimensioned to receive and guide the core rod 29.

Away from the proximal end of the collar, the diameter of the annulus is substantially greater than that of the core rod to provide a housing 34 for a biasing means such as spring 35. The coils of spring 35 encircle the core rod. Although a coil spring 35 is a preferred biasing means, biasing can be accomplished by other means, such as a stack of Belleville washers or an elastic insert.

Spring 35 or other biasing means engages retaining ring 36 mated to core rod 29. Retaining ring 36 can be mated to the core rod by clamping engagement with a circumferential groove 37 in the rod. The retaining ring can be a conventional C-clip which engages the groove, or it can be a clamp or any other structure against which the biasing means can exert a biasing force and which is restrained from movement relative to core rod 29 in a direction parallel to the long axis of the core rod.

As illustrated, an annular locking bolt 38 engages internal threads 39 at the distal end of collar 32. The bore 40 through locking bolt 38 is dimensioned to receive and, in conjunction with the annulus at the proximal portion of the collar, to restrain motion of core rod 29 to axial movement. Locking bolt 38 also retains and can compress the biasing means. Core rod 29 is biased in the direction of the die table when the collar is installed on stationary mounting point 21 and is retained in slidable engagement with collar 28 by retaining ring 36 and locking bolt 38. The height of rod tip 41 is adjusted by advancing or retracting collar 28 relative to stationary mounting point 21, e.g. by rotating the collar when in threaded engagement with the stationary mounting point.

Core rod 29 can vary in diameter along its length. A preferred diameter of rod tip 41 is from about 0.5 mm to about 10 mm, more preferably about 4.5 mm. However, for rigidity, the core rod should be thicker, preferably from about 4 mm to about 12 mm throughout most of its length, more preferably about 9 mm. The rod can taper gradually from a narrow diameter at the tip to a larger shank diameter or it can change abruptly at a shoulder 42.

The core rod can be of two-piece construction. For instance, the core rod tip 41 could be adapted to attach to the core rod by providing external threads at its lower end and a socket with internal threads at the upper end of the core rod, or vice versa. A two-piece construction allows the core rod tip to be replaced if it is damaged or if a core rod tip of a different shape is desired. The core rod tip can have any desired diameter or shape.

Punch assembly 27 further comprises annular punch 30. Annular punch 30 is provided with means for attaching to lower reciprocating ram 20, such as locking flat 43. The bore 44 through annular punch 30 is dimensioned to receive and surround core rod 29 while permitting axial movement of annular punch 30 independent of the core rod. The bore through annular punch 30 can vary in diameter along the length of the punch providing an annular flange 45 for engagement with shoulder 42 on the core rod. Engagement of flange 45 with shoulder 42 prevents annular punch 30 and collar 28 from abutting each other during handling and installation. Annular punch contact surface 46 presses against the powder or granular material during compression. Contact face 46 can have any desired contour, e.g. standard concave, deep concave, extra deep concave, modified ball or flat. Preferably contact face 46 is flat with a beveled edge for ease of ejection of the finished tablet.

The columnar punch, annular punch, core rod and collar are preferably made of metal, more preferably steel, most preferably stainless steel.

In the final dosage form with recessed core tablet, the depth of first void 13 (FIG. 1) is determined by the height of protrusion 25 . The depth of second void 14 is determined by the fill depth, strength of the bias on the core rod, the compressibility of the material and the thickness of the core tablet. These parameters can be adjusted by routine experimentation to control the depth of second void 14, which is suitably commensurate with the depth of first void 13.

In a second dosage form embodiment, either one or both of opposed surfaces 3 and 4 of the core tablet are flush with the annular faces 6 and 7 of the annular body 2. This alternative embodiment can be produced by using a columnar punch as previously described but lacking a protrusion 25. Surface 3 will generally be flush with annular face 6 if the columnar punch has a flat contact face. Whether the opposed surface 4 is flush with annular face 7 will depend on the fill depth, compressibility of the powder or granular material and thickness of the core tablet, which factors can be adjusted by routine experimentation to yield a dosage form with surface 4 recessed the desired distance relative to annular face 7.

To further illustrate the invention and the operation of the toolset, a cycle of operation will now be described. The cycle of operation is embodied in a process that constitutes a third aspect of the invention.

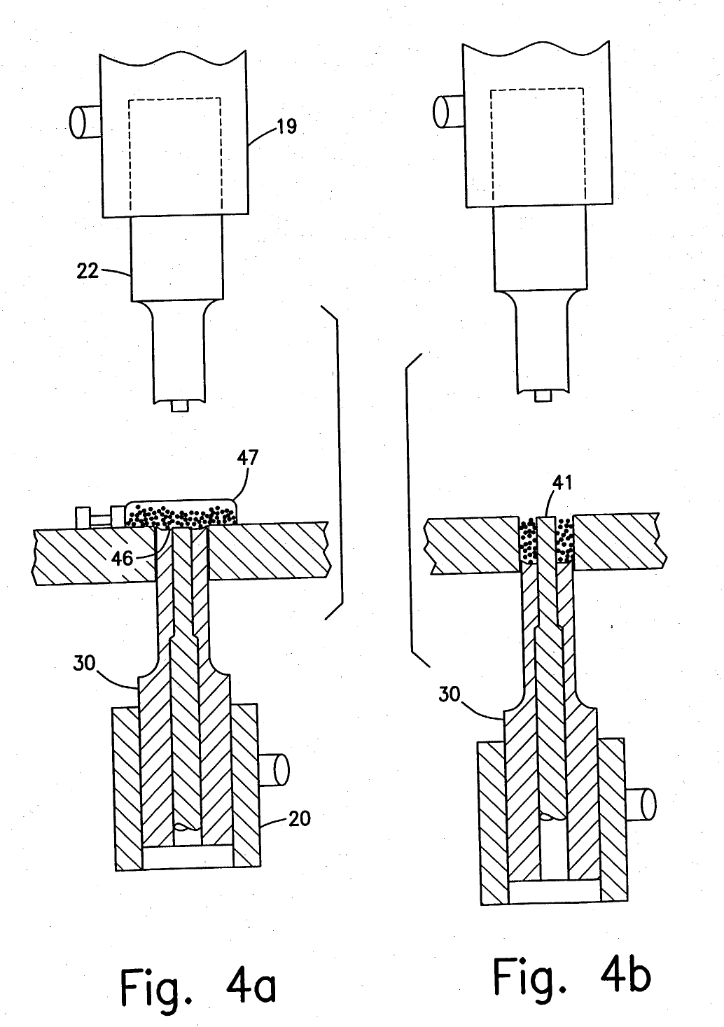

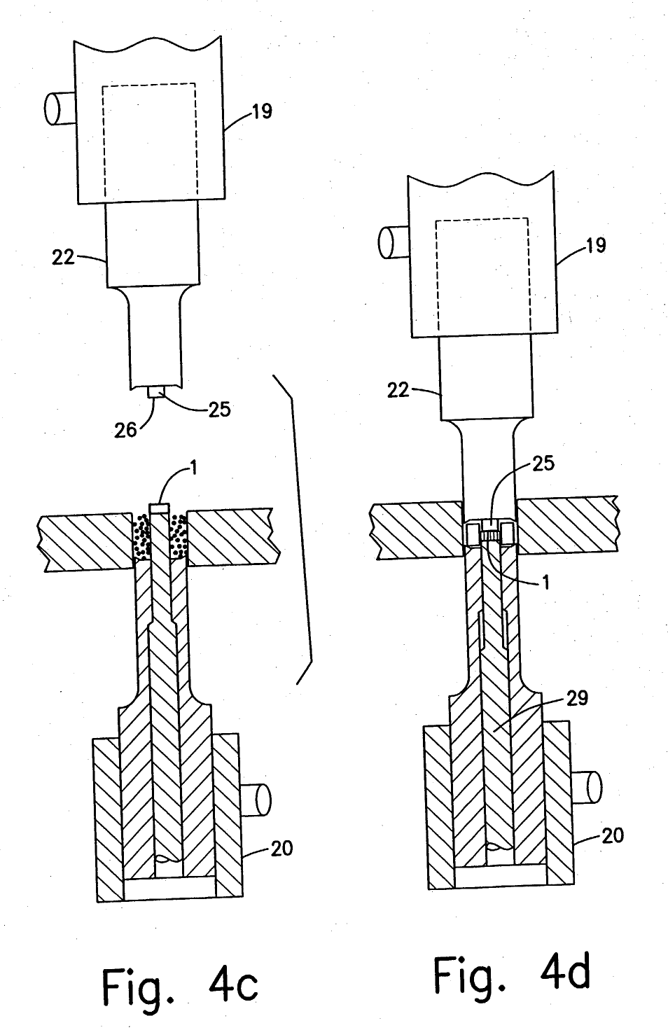

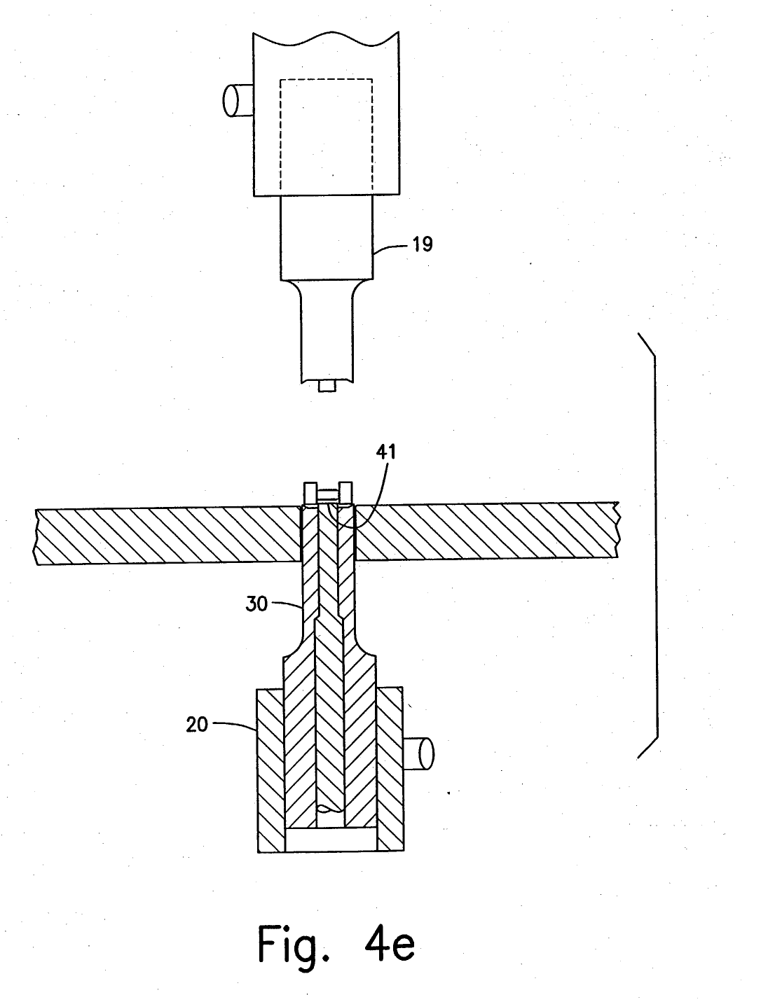

The cycle of operation is first illustrated on a single station press. The cycle begins with the first action that occurs after ejection of the tablet formed in a previous cycle. Referring now to FIG. 4a, feed shoe 47 moves laterally over the die bore while the annular punch 30 is in an advanced position such that contact surface 46 is substantially flush with the top surface of the die. In so doing, the feed shoe sweeps a finished tablet from atop the annular punch toward a chute leading to a receptacle where the tablets are collected. Annular punch 30 is retracted while the tip 41 of core rod 29 remains flush with the die surface (FIG. 4b). Retraction of the annular punch causes an annular cavity to form into which particles of the powder or granular material are fed from the feed shoe by gravity and/or pressure differential. Once the cavity is filled, the feed shoe is shifted away from the die bore.

Pre-compressed core tablet 1 is positioned atop the core rod using any conventional apparatus for producing tablets with a compressed coating such as that of a Kilian RUD press (FIG. 4c). The positioning means forms no part of the invention and has been omitted for clarity.

Columnar punch 22 is advanced by upper reciprocating ram 19 (FIG. 4d). As columnar punch 22 approaches the bore, the raised surface 26 of protrusion 25 presses upon core tablet 1. As columnar punch 22 enters bore 17, core tablet 1 is pushed into the bore by the protrusion against the biasing force exerted on core rod 29. Continued movement of columnar punch 22 into the die bore compresses the powder or granular material into an annular body around the core tablet. Strong compressive forces can be exerted on the powder or granular material without breaking the core tablet because the core tablet travels into the bore before the powder or granular material is fully compressed.

Those skilled in the art may also appreciate that protrusion 25 could be replaced with a core rod in the columnar punch that is biased toward an extended position so that the tip of the rod would press against core tablet 1 during compression. Such a core rod for the columnar punch would not necessarily be attached to a stationary mounting point on the press. It would be biased with greater force than core rod 29 so that pressure exerted by the columnar punch would push the core tablet into the bore against the resistance of the core rod.

After the powder or granular material is compressed, the columnar punch is withdrawn. Either concurrently or subsequently, annular punch 30 is advanced by lower reciprocating ram 20 to a position such that contact face 46 is substantially flush with the upper surface of the die to elevate the finished tablet above the die where it can be swept from the die table in a subsequent cycle of operation (FIG. 4e). Meanwhile, the core rod is biased back to its original position flush with the die surface.

The toolset is well adapted for use in a rotary tablet press. The cross-sectional dimension and shape of the columnar punch, and the dimensions and shape of the protrusion (if present) are the same as in a punch adapted for use in a reciprocating tablet press. The other dimensions of the toolset are generally dictated by the dimensions and layout of a particular tableting press. These dimensions can be readily determined by those skilled in the art. The cross-sectional dimensions and shape of the annular punch and of the core rod are the same as in a punch adapted for use in a reciprocating tablet press, again with other dimensions being dictated by the dimensions and layout of a particular tableting press. These dimensions can be readily determined by those skilled in the art. In addition, the punches include conventional bearing surfaces at the end distal to their contact surfaces for engaging the cams and rollers that control their motion along the axis of the die bore, such as those shown in the patents that are incorporated by reference below.

In an annular punch for use in a rotary machine, the core rod biasing means preferably is housed in the annular punch and includes a means for adjusting the degree of extension of the core rod and/or the bias, such as a set screw or similar device.

Conventional rotary tablet presses are well known in the art. Some rotary presses and improvements related thereto are described in U.S. Pat. Nos. 5,462,427, 5,234,646, 5,256,046 and 5,635,223, which are incorporated herein by reference in their entirety. Rotary presses have a moving die table that rotates around a vertical axis. Mounted above and below the die table are upper and lower punch carriers that rotate synchronously with the die table. The punch carriers can be generally drum shaped bodies of about the same diameter as the die table or they can have arms that extend outward from a lesser diameter ring. The punch carriers are provided with a plurality of vertical holes or slots at regular intervals around their circumference or through the ends of the arms. When the press is in operation, punches are inserted into each slot with their contact faces pointing toward the die table. Each punch has a bearing means at the end opposite the contact face. The bearing means engage stationary cams and rollers which control the vertical motion of each punch during a cycle of operation. The cams and rollers are arranged such that in a cycle of operation, a powder or granular material is fed into a die while the lower punch is inserted into the die. Pressure is applied to the powder or granular material to produce a compressed body. After compression, one or more of the punches is removed from the die and the dosage form is released. Rotary presses are especially suited for high volume production because they typically contain numerous punch and die sets operating simultaneously.

A cycle of operation using the toolset of this invention adapted for use in a rotary press will now be described. As the die table rotates, one of the dies passes under a fill shoe or force feeder. While the die is passing underneath the shoe or feeder, the annular punch is withdrawn by the cam. The core rod remains in an extended position, up to the upper die face. The annular space left by withdrawal of the annular punch is filled with powder or granulate. At the next station, a core tablet is inserted onto the tip of the core rod by conventional means, such as those used in “press coat” machines like the Kilian RUD. The core tablet can be positioned atop the core rod by any method. On further rotation, the die comes to the compression station where the columnar punch with, or without, its protrusion moves downward and pushes the core tablet into the bed of powder or granular material. The force of the columnar punch retracts the core rod against the bias and the powder or granular material is compressed into an annular shape around the core tablet. In the dosage form product, one recess is defined by the height of the protrusion and the other recess is defined by a combination of the factors such as the strength of the bias, the fill depth, the compactability of the powder or granular material and the thickness of the core tablet. After the powder is compressed, the die rotates further to where the columnar punch is withdrawn from the die. Either concurrently or subsequently, the annular punch is raised until it reaches the die face. The core rod rises concurrently to the die face due to the bias. The tablet is swept out of the die by an ejection element and is collected.

While reference has been made to “upper” and “lower” elements in the description of the toolset and process for making solid dosage form according to the invention, the spacial relationships of the elements are determined by the design and construction of the press in which they are used. Use of the terms “upper” and “lower” is not intended to limit the invention to a vertical arrangement of the elements.

Having thus described the present invention with reference to certain preferred embodiments, the invention will now be further illustrated by the following example.

### EXAMPLES

### Example 1

### Immediate Release Monosodium Alendronate Tablets

This example summarizes a study designed to determine the rate and extent of absorption of alendronate sodium in human subjects upon administration of a solid pharmaceutical dosage form of the present invention (“protected tablet”).

### Materials and Methods

Protected tablets were made as follows.

Tablet Core: 85.4 g of alendronate trihydrate (TEVA Assia Ltd.) and 2.6 g of xylitol (Danisco Sweeteners OY) were granulated with 20 g water in a Diosna (model P1/6) granulator for 3 min. The granulate was dried at 40° C. for one hour in a fluidized bed dryer and milled through a 0.8 mm screen. The granulate was blended with 11 g crospovidone NF (BASF Pharma) for five minutes. One gram magnesium stearate NF/EP (Mallinkrodt Inc.) was added and the granulate was further blended for an additional 0.5 minutes. The blend was compressed using a Manesty F3 single punch tablet machine fitted with a 5 mm flat beveled punch. The tablet weight was 94.9 mg±1.0% RSD. The hardness of the core tablets was 3-6 kP.

Protected Tablets: A mixture of 94 grams compressible sucrose (Nu-Tab™, DMV International) and 5 grams microcrystalline cellulose (Avicel™ pH102, FMC International) were blended for five minutes. One gram magnesium stearate (NF/EP, Mallinkrodt Inc.) was added and the mixture was blended for another half a minute.

A Manesty f3 single punch tableting machine was fitted with a spring-biased columnar punch and punch assembly constructed in accordance with the present invention. The core rod was designed for a 5 mm round core tablet and the die and punches for the outer tablet were designed to produce a round, 9 mm diameter, flat beveled solid pharmaceutical dosage form. The upper punch had a protrusion of diameter 4.5 mm and 1.2 mm height. The tablet press was operated and the protected tablets were produced. The tablet weight was 474 mg±0.62% RSD and the hardness of the protected tablets was 12-15 kP. The alendronate trihydrate content, expressed as alendronic acid was 66.8 mg±1.38% RSD (82.4 mg alendronate trihydrate being equivalent to 70 mg alendronic acid).

The drug-containing inner tablet was recessed from the surface of the annular body by about 1 mm.

### Pharmacokinetic Study

A clinical trial involving twelve (12) human volunteers was conducted to demonstrate the pharmacokinetics of a solid dosage form of the present invention containing 70 mg alendronate. Its pharmacokinetics was compared to that of a commercial 70 mg Fosalan™ tablet of the prior art (Merck, Sharpe & Dohme).

### Method

The study was a randomized, open-label, 2-treatment, 2 period, 2 sequence crossover design under fasting conditions. Twelve (12) healthy adult male volunteers, 18-55 years of age were the subjects in the study.

The study was divided into first and second study periods, each of 36 hours duration, with a 14 day “wash-out” period between the study periods. All subjects who completed both study periods were included in the analysis. Subjects were randomly assigned to two groups. One group was administered alendronate via the protected tablet in the first period and administered control Fosalan in the second period. The order of administration to the second group was reversed.

In both periods, alendronate was administered in the fasted state. A standardized meal was provided 4 hours after administration. Snacks were provided on a standardized schedule that was the same for all subjects in both study periods. Water was provided ad libitum. In addition, subjects were encouraged to drink at least 200 ml of water at regular intervals during each study period.

The bioavailability of alendronate was determined by measuring the cumulative levels of alendronate excreted in the urine over a 36 hour period following oral ingestion of the test and control tablets (hereafter “Ae₀₋₃₆”). An initial (t=0) urine sample was taken immediately after administration. Urine samples were taken at 11 regularly scheduled points in time over the 36 hour test period. All urine samples were analyzed for alendronate using a validated HPLC-FLR assay.

### Results

The main pharmacokinetic parameters obtained from the analyses of urine samples are collected in Table 1.

TABLE 1
Pharmacokinetic Parameters

| | Administration via | | | Administration via | | |
|---|---|---|---|---|---|---|
| | Protected Tablet | | | Fosalan (control) | | |
| Parameter | Mean | ±SD | CV (%) | Mean | ±SD | CV (%) |
| Ae₀₋₃₆ (μg) | 113.6 | 77.2 | 67.9 | 102.6 | 36.8 | 36.8 |
| Rₘₐₓ (μg/h) | 37.9 | 19.9 | 51.5 | 31.7 | 11.8 | 38.3 |
| Tₘₐₓ (h) | 1.4 | 0.9 | — | 1.4 | 0.9 | — |

A comparison of the pharmacokinetic parameters of the dosage form in accordance with this invention with the pharmacokinetic parameters of the prior art dosage form is provided in Table 2.

TABLE 2
Comparison of Pharmacokinetics of the Protected Tablet to Prior Art

| | Ae₀₋₃₆ (mg) | Rₘₐₓ (mg/h) |
|---|---|---|
| Geometric Mean of Ratio | 0.99 | 1.12 |
| 90% Geometric C.I. | 75.31% to 128.79% | 93.98% to 135.01% |
| Intra-subject C.V. | 37.48% | 24.85% |

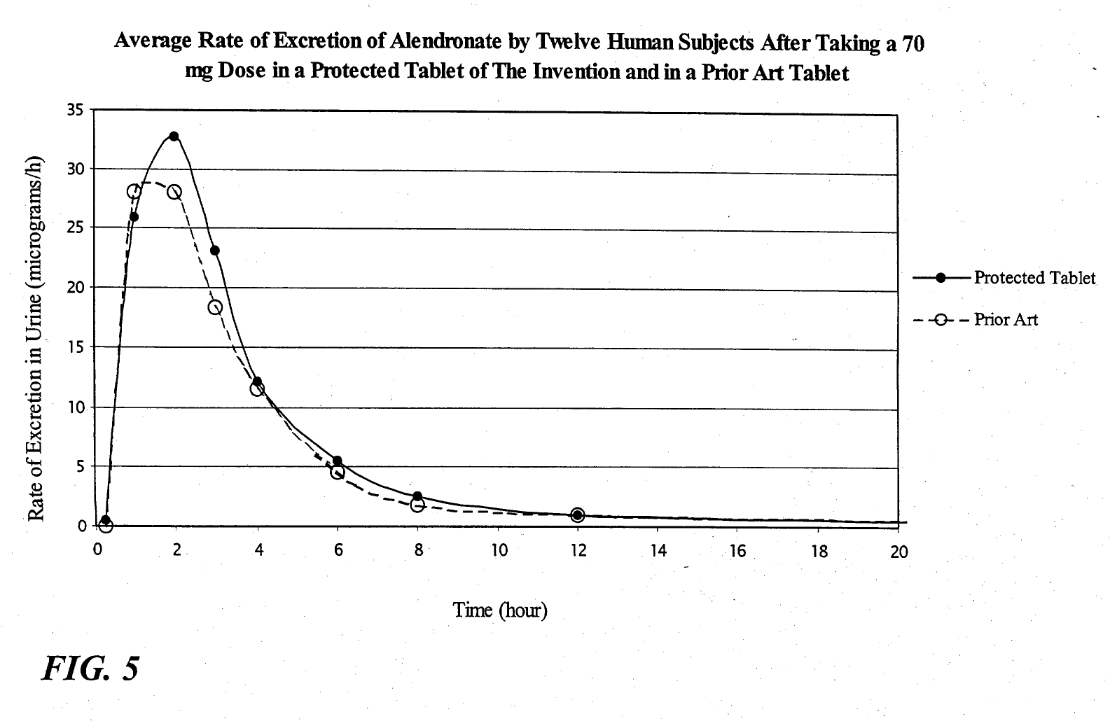

By reference to Tables 1 and 2, and FIG. 5, one can see that alendronate administered via the solid dosage form of the present invention gives essentially the same pharmacokinetic results as administration via Fosalan. The total amount of the alendronate excreted into urine over 36 hours is essentially the same for both treatments with the maximum rates of excretion (parallel to Cₘₐₓ in a pharmacokinetic study of plasma levels of drug) also close.

The profile of excretion into urine was similar for all subjects and in both treatments. The majority of the subjects had their maximum rate of excretion (Rₘₐₓ) between one and two hours. For five of the subjects, the Rₘₐₓ occurred earlier than 1 hour after administration when they took Fosalan. Four of the subjects experienced a Rₘₐₓ in less than an hour when they took the protected tablet. One of the subjects had an Rₘₐₓ in the third hour when he took Fosalan while two of the subjects had a Rₘₐₓ in the third hour when they took the protected tablet.

The total amount of excreted alendronate ranged from 36.9 μg to 158.6 μg when Fosalan was administered and from 30.1 μg to 284.4 μg when the solid oral dosage form of the present invention was administered. In only two subjects was there a greater than two fold difference between the total amount of excreted alendronate between the two treatments. Another subject excreted a very low amount of alendronate regardless of how the alendronate was administered.

The bioavailability of alendronate administered via the novel solid dosage form of the present invention is equivalent to that of alendronate administered by dosage forms of the prior art. However, the dosage form of the prior art does not provide any protection against contact of the alendronate with the mucous membranes of the esophageous and stomach while the bioequivalent novel dosage form of the present invention affords such protection.

### Drug Release Profile

Dissolution was measured in a USP apparatus III dissolution unit (Hanson B-3) unit at 37° C. The alendronate content of samples taken at 5, 10, 15 and 30 minutes was determined by HPLC on an anion column using refractive index detection. The results of the dissolution are reported in Table 3.

TABLE 3

| | Cumulative Percent |
|---|---|
| Time (m) | Release |
| 5 | 48 |
| 10 | 70 |
| 15 | 85 |
| 30 | 98 |

The outer mantle took more than one hour to dissolve.

The tablets were tested in a human pharmacokinetic study and shown to be bioequivalent to commercially available alendronate (70 mg).

### Example 2

### Extended Release (Zero Order Release) Oxybutynin Tablets

The annular coated tablet is uniquely fit for extended controlled release, particularly when one needs to approximate zero order release over an extended period of time. The drug is delivered through the exposed axial faces of the delivery system. These faces retain a constant cross-section during drug delivery, thereby aiding in the achieving of a constant rate of drug release.

### A. Inner Tablet

Oxybutynin (50 g), was mixed with anhydrous lactose (50 g) in a Zanchetta Rotolab™ one pot granulator. The granulation solution, 5% w/w hydroxypropylcellulose (Klucel™ LF, 21 ml), was added with stirring at 500 rpm until thorough mixing was achieved. The granulate was dried in the one pot granulator at 45-50° C. with gas stripping for a time of about 20 minutes. The granulate was milled in a Quadro Comil™ milling machine using a screen size of 1143 μm.

The oxybutynin granulate (27.6 g), was mixed with hydroxypropylmethylcellulose, (HPMC, Methocel™ K15M, 19 g), and compressible sucrose (Nu-Tab™, 52.4 g). Magnesium stearate, 1 g, was added with mixing. The blend was compressed into tablets on a Manesty f3 single punch tablet machine using 6 mm flat beveled punches to produce tablets weighing about 110 mg and having a hardness of 4 Kp.

### B. Non Dissolving Outer Mantle on Cylindrical Surfaces

Polyethylene glycol (PEG 4000) was milled and passed through a 500 μm screen. The milled PEG 4000 (24 g), was mixed with polyvinylpyrrolidone (Povidone™, PVP K-30, 5 g), and ethylcellulose (Ethocel™ 7 cps, 71 g), for 3 minutes. Magnesium stearate (1 g), was added and the blend mixed for another 0.5 minutes. The inner cores, produced above, were pressed within the outer mantle using this blend and a 9 mm outer cylinder spring loaded core rod tooling previously described. The core rod diameter was 4.5 mm. The upper punch had a protrusion of 5 mm diameter tapering to 4.5 mm at its upper surface with a height of 1.2 mm. The final product, an annular ring coated tablet with recessed exposed axial faces, had an outer diameter of 9 mm, a total weight of 350 mg and contained 15 mg oxybutynin (Formulation A).

### C. Drug Release Profile

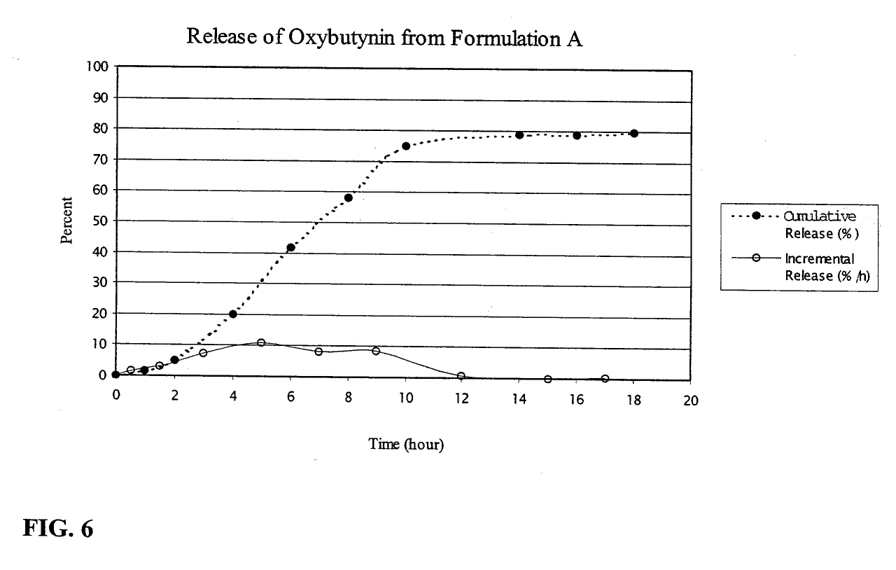

The drug release profile of oxybutynin from the delivery system of Example 1 was tested in an USP apparatus II dissolution tester using 900 ml of phosphate buffer pH=6.8 at 37° C., 50 rpm. The oxybutynin content of the samples were determined by an HPLC method with UV detection. The results are reported in Table 4, below, and presented graphically in FIG. 6.

TABLE 4

| | Cumulative Percent |
|---|---|
| Time (h) | Release |
| 1 | 1.7 |
| 2 | 4.9 |
| 4 | 20.0 |
| 6 | 41.8 |
| 8 | 58.3 |
| 10 | 75.1 |
| 14 | 79.0 |
| 16 | 79.1 |
| 18 | 79.5 |

### D. Control of the Release by Changes in the Inner Tablet Formulation

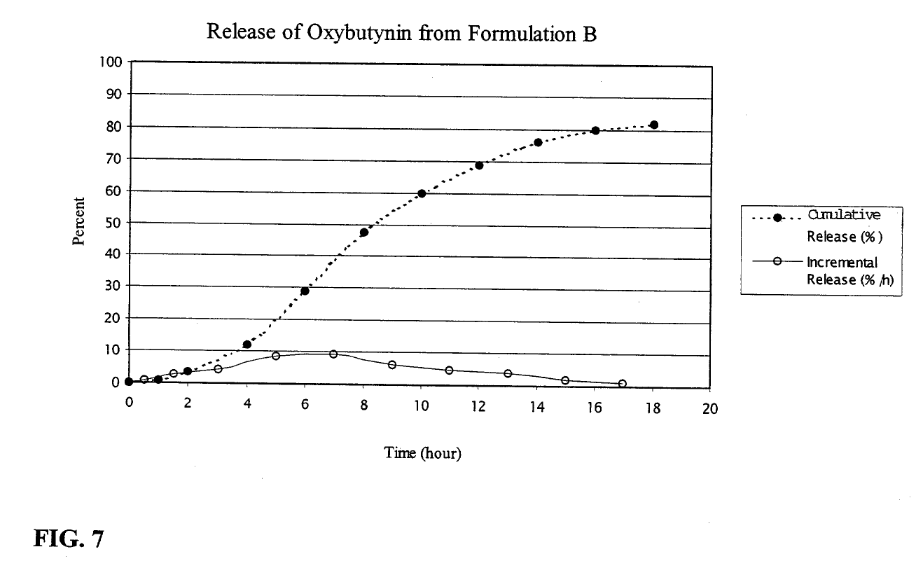

The above procedure for the preparation of the inner tablet was repeated, using 30 g of Methocel™ K15M and 41.4 g of Nu-Tab™, thus raising the content of the gel forming HPMC and lowering the content of the dissolving sucrose (Formulation B). The results of the dissolution experiment are reported in Table 5, below, and depicted in FIG. 7.

TABLE 5

| | Cumulative Percent |
|---|---|
| Time (h) | Release |
| 1 | 0.8 |
| 2 | 3.4 |
| 4 | 11.8 |
| 6 | 29.1 |
| 8 | 47.5 |
| 10 | 59.8 |
| 12 | 68.8 |
| 14 | 76.2 |
| 16 | 79.8 |
| 18 | 82.0 |

A significant slowing of drug release in the first ten hours was observed.

### E. Control of Release by Changes in the Formulation of the Outer Mantle

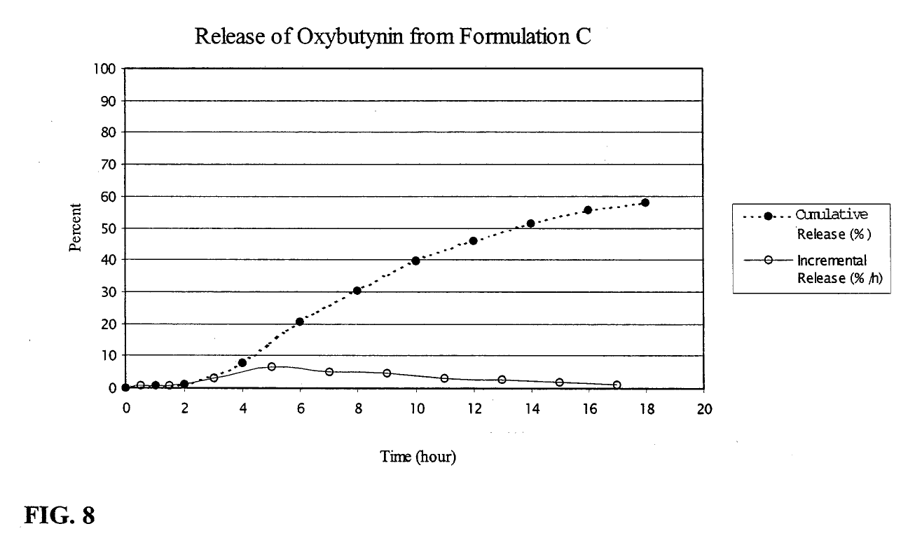

The procedure for the preparation of Formulation B was repeated, with the outer mantle containing 14 g of PEG 4000 and 81 g of Ethocel™ (Formulation C). The results of the dissolution experiment are shown in Table 6, below, and depicted graphically in FIG. 8.

TABLE 6

| | Cumulative Percent |
|---|---|
| Time (h) | Release |
| 1 | 0.6 |
| 2 | 1.2 |
| 4 | 7.6 |
| 6 | 20.5 |
| 8 | 30.5 |
| 10 | 39.6 |
| 12 | 46.1 |
| 14 | 51.5 |
| 16 | 55.5 |
| 18 | 58.0 |

Again, significant changes in the rate of drug release were observed, demonstrating that changes in the formulation of the inner core tablet or the outer annular body can determine the rate of release of active drug material.

### Example 3

### Fast Dissolving Tizanidine Tablets for Sublingual Delivery

Sublingual tablets were formed into an inner core of a fast disintegrating formulation containing tizanidine (2 mg) and an outer annular ring of protective excipients.

### A. Inner Tablet

The inner cores were made by mixing tizanidine hydrochloride (4.5 parts) and crospovidone (20 parts), for 2 minutes. Sodium saccharin (0.5 parts), MicrocelLac100™ (73.6 parts), and menthol (0.4 parts) were added and the mixing continued for 3 more minutes. Magnesium stearate (1 part) was added and the mixing continued for a half a minute. The mixture was compressed on a Manesty f3 tablet press fitted with a five mm flat beveled punch. The tablets formed were of 5 mm diameter, weighed 45 mg each, were about 2 mm thick and had a hardness of 1-3.5 Kp.

### B. Dissolving Outer Mantle

The outer annular ring was made by mixing Nu-Tab™ (48.5 parts), MicrocelLac100™ (a 25:75 mixture of microcrystalline cellulose and lactose commercially available for direct compression, 45 parts), sodium saccharin (0.5 parts) and of crospovidone (5 parts) for 5 minutes. Magnesium stearate (1 part) was added and the mixing continued for a half a minute. The mixture was compressed on a Manesty f3 tablet press fitted with the spring loaded core rod tooling as previously described. The entire tablet weight was 290 mg, the outer diameter was 9 mm, the tablet height about 4.5 mm and the hardness 5-9 Kp.

### C. Drug Release Profile

The tablets were tested for total disintegration of the inner tablet in 3 ml water within 4 minutes and at least 85% dissolution of the tizanidine in 450 ml water at 37° C. and 50 rpm in a USP apparatus II dissolution system in 15 minutes. The outer mantle dissolves after about 15 minutes.

### Example 4

### Release of Two Drugs at Different Rates

The annular body and core tablet can be formulated to contain different drugs and to release the drugs with totally different release profiles. The rates of release can be controlled by the formulation of the core tablet and annular ring and by their geometries. In this case,we have formulated a carbidopa immediate release profile in the core tablet with controlled release of levodopa from the annular body while using an oval tablet as the annular ring around either a cylindrical tablet or an inner oval tablet. The inner cores, both cylindrical and oval, were themselves hollow with a cylindrical hole in each of them.

### A. Inner Tablets

Carbidopa (160 g) was mixed with pre sieved (500 μm screen) xylitol (40 g) in a Diosna p1/6 granulator. Water (45 ml) was added as the granulation solution. The mixture was granulated for 5 minutes at 500 rpm and further massed at 800 rpm for 1.5 minutes. The granulate was air dried at room temperature overnight and then milled, while still wet, through a 1.6 mm screen. The milled granulate was dried in a fluidized bed for 30 minutes at 40° C. and then milled through a 0.8 mm screen. This granulate, 56.3 g, was mixed with crospovidone (10 g) and MicrocelLac100™ (32.7 g) for three minutes. Magnesium stearate (1 g) was added to the blend which was further mixed for 0.5 minutes. The blend was compressed in a Manesty f3 single punch tableting machine using three different core rod punches to make hollow cylinders of the following dimensions:

Formulation D: cylindrical outer diameter 7.5 mm inner diameter 2.5 mm

Formulation E: cylindrical outer diameter 7.0 mm inner diameter 4.6 mm

Formulation F: oval outer diameters 12×6 mm, inner diameter 3 mm.

Each tablet contained 54 mg carbidopa.

### B. Drug Containing Non Dissolving, Oval Outer Mantle

Levodopa (150 g) was mixed with xylitol (75 g) and hydroxypropylcellulose (Klucel™ LF, 25 g) at 500 rpm for 5 minutes. Ethanol (50 ml) was added slowly and the granulate was formed at 500 rpm over 1.5 minutes. The granulate was air dried overnight at room temperature and milled through a 0.8 mm screen.

The levodopa granulate (44.4 g) was mixed with ethylcellulose (Ethocel™ 7 cps, 30 g) and Cellactose 80™ (25:75 mixture of powdered cellulose: lactose for direct compression, 24.6 g) for three minutes. Magnesium stearate (1 g) was added and the blend mixed for another 0.5 minutes.

The previously formed inner tablets, Formulations D, E and F were compressed in an oval shaped mantle core on their radial surfaces using an oval shaped spring loaded core rod punch as previously described, with dimensions 17.6×8.8 mm with an internal core rod of 5 mm diameter and an upper punch with a protrusion of 5 mm diameter tapering to 4.5 mm at its height of 1.8 mm. The total weight of each tablet was 750 mg and each contained 200 mg of levodopa.

### C. Drug Release Profile

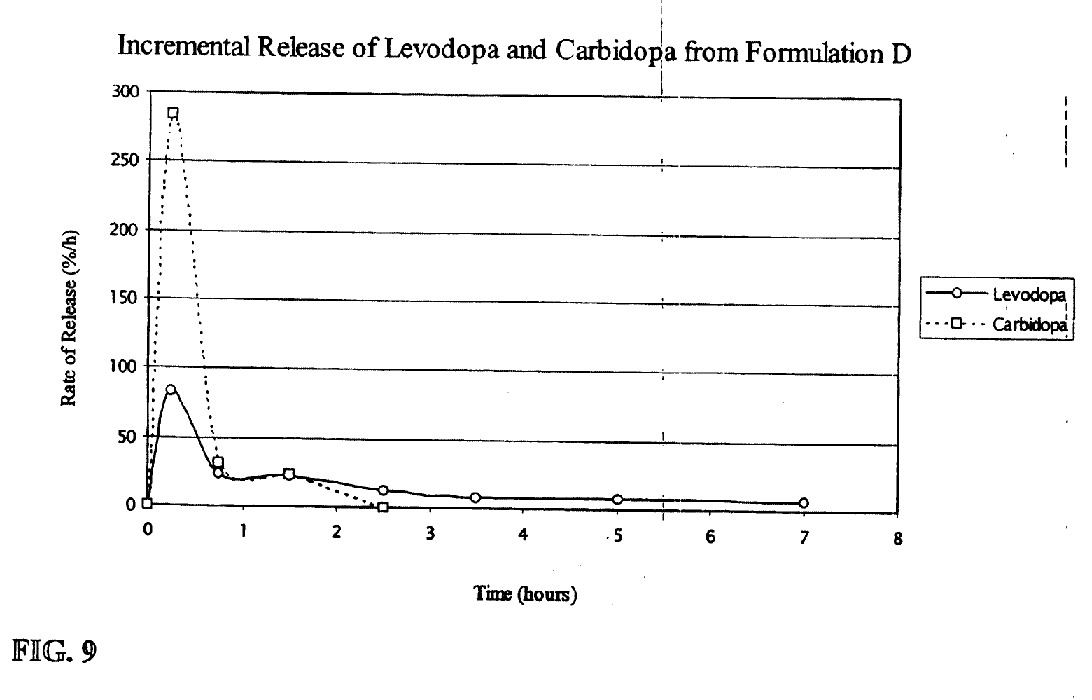

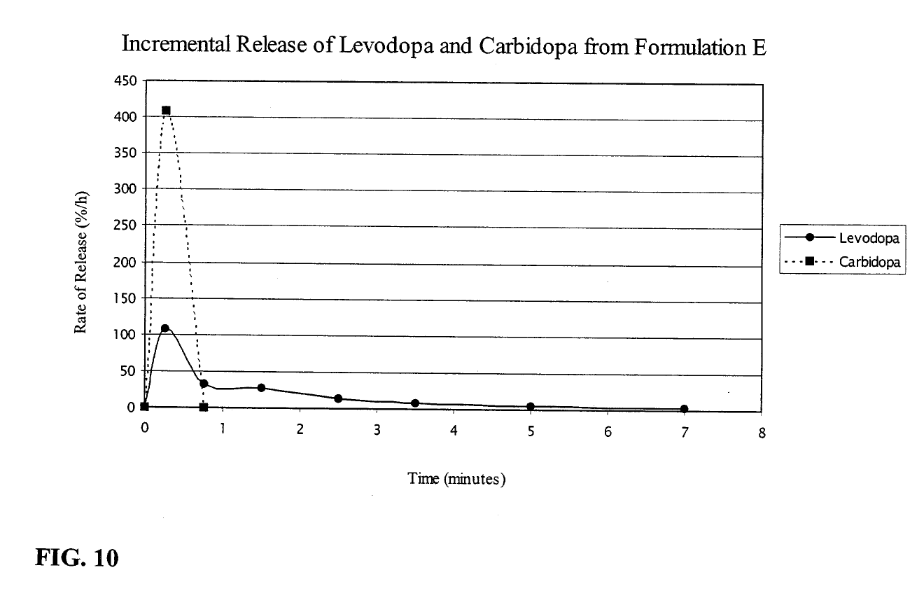

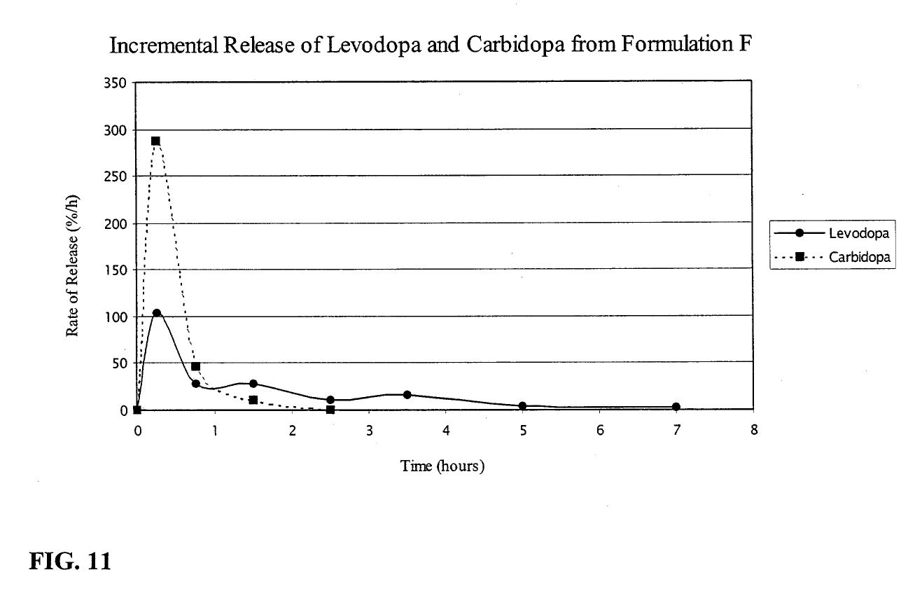

Dissolution was carried out in 0.1N HCl (900 ml) at 37° C. in a USP Apparatus II dissolution tester at 50 rpm and the levodopa and carbidopa concentrations of each sample were determined by HPLC. The results of the dissolution experiments are provided in Tables 7, 8 and 9 and depicted in FIGS. 9, 10, and 11.

TABLE 7
Dissolution Results for Formulation D

Cumulative Percent Release

| Time (h) | Levodopa (%) | Carbidopa (%) |
|---|---|---|
| 0.5 | 21 | 71 |
| 1 | 33 | 87 |
| 2 | 50 | 105 |
| 3 | 62 | |
| 4 | 70 | |
| 6 | 81 | |
| 8 | 94 | |

TABLE 8
Dissolution Results for Formulation E

Cumulative Percent Release

| Time (h) | Levodopa (%) | Carbidopa (%) |
|---|---|---|
| 0.5 | 27 | 102 |
| 1 | 43 | |
| 2 | 63 | |
| 3 | 76 | |
| 4 | 85 | |
| 6 | 94 | |
| 8 | 101 | |

TABLE 9
Dissolution Results for Formulation F

Cumulative Percent Release

| Time (h) | Levodopa (%) | Carbidopa (%) |
|---|---|---|
| 0.5 | 26 | 72 |
| 1 | 40 | 95 |
| 2 | 61 | 103 |
| 3 | 72 | |
| 4 | 88 | |
| 6 | 93 | |
| 8 | 99 | |

Thus, two drugs with totally different release profiles can be delivered with independent control of the rate of release of each drug. It should be noted that this control can be achieved by shaping and sizing the core tablet, e.g. by providing it with hole of predetermined size or shape, without necessitating a change in formulation.

### Example 5

### Annular Coated Tablet for Taste Masking

### A. Inner Tablets

Sumatriptan succinate (70 parts), is granulated in water (20 parts) with microcrystalline cellulose (Avicel™ PH 101, 80 parts). The granulate is dried in a fluidized bed drier for 30 minutes at 40-50° C. and subsequently milled through a 0.8 mm screen. The granulate (75 parts) is mixed with anhydrous lactose (9 parts), microcrystalline cellulose (Avicel™ PH101, 10 parts) and croscarmellose sodium (AC-DI-SOL™, 5 parts) for 3 minutes. Magnesium stearate (1 g) is added and the blend mixed for another 0.5 minutes. Tablets are pressed on a Manesty f3 single punch tableting machine using a 6 mm flat beveled punch. The tablet weight is 100 mg and contains the equivalent of 25 mg sumatriptan.

### B. Dissolving Outer Mantle

A mixture of compressible sucrose (Nu-Tab™, 94 g), microcrystalline cellulose (Avicel™PH102, 5 g), and of menthol (1 g) are blended for five minutes. Magnesium stearate (1 g) is added and the mixture is blended for another half minute.

Tablets are formed using the inner cores described in Example 4, above, and a 9 mm outer cylinder spring loaded core rod tool previously described. The tablets obtained are cylindrical tablets of 9 mm outer diameter with the axial faces uncoated and recessed from the surface. The tablet weigh a total of 475 mg.

### C. Drug Release Profile

The release profile of the tablets is measured in a USP Apparatus II Dissolution tester in 900 ml water at 37° C. and 50 rpm. The tablets are expected to provide a drug release of greater than 80% in 30 minutes.

Having thus described the invention with reference to certain preferred embodiments, other embodiments will be apparent from this description to those skilled in the art to which the invention pertains. It is intended that the specification is considered exemplary only, with the scope and spirit of the invention being indicated by the claims which follow.

What is claimed is:

1. A pharmaceutical dosage form for oral administration to a patient comprising a core tablet containing an active ingredient sheathed in a annular body of compressed powder or granular material, that releases the active ingredient from the core tablet at a rate in the range of from 3% per hour to 12% per hour over a period of seven hours or more.

2. The pharmaceutical dosage form of claim 1 wherein the active pharmaceutical ingredient is selected from the group consisting of oxybutynin, alendronate, carbidopa, levodopa, tizanidine, sumatriptan and pharmaceutically acceptable salts and solvates thereof.

3. The pharmaceutical dosage form of claim 2 wherein the active pharmaceutical ingredient is oxybutynin.

4. The pharmaceutical dosage form of claim 1 wherein the core tablet further contains hydroxypropylmethylcellulose and compressible sugar.

5. The pharmaceutical dosage form of claim 1 wherein the annular body contains a solid polyethylene glycol, polyvinylpyrrolidone and ethyl cellulose.

6. The pharmaceutical dosage form of claim 5 wherein the polyethylene glycol is polyethylene glycol 4000.

7. The pharmaceutical dosage form of claim 1 wherein the rate of release of oxybutynin from the core tablet is measured in a United States Pharmacopeia standard apparatus II solution tester in an aqueous solution buffered at 6.8 at 37° C. with a stirring rate of 50 revolutions per minute.

8. The pharmaceutical dosage form of claim 1 wherein the active ingredient is released from the core tablet at a rate in the range of from 3% per hour to 12% per hour over a period of ten hours or more.

9. A pharmaceutical dosage form for oral administration to a patient comprising a core tablet containing an active ingredient sheathed in an annular body of compressed powder or granular material, that releases about 90% or more of the active ingredient from the core tablet within about 30 minutes.

10. The dosage form of claim 9 wherein the rate of release is measured in a United States Pharmacopeia standard apparatus III dissolution unit at 37° C.

11. The dosage form of claim 9 wherein the active ingredient is monosodium alendronate.

12. The dosage form of claim 11 wherein about 85% of the monosodium alendronate is released within about 15 minutes.

13. The monosodium alendronate dosage form of claim 11 wherein the core tablet further contains xylitol and crospovidone.

14. The monosodium alendronate dosage form of claim 11 wherein the annular body contains compressible sucrose and microcrystalline cellulose.

15. A pharmaceutical dosage form for oral administration to a patient comprising a core tablet containing an active ingredient sheathed in an annular body of compressed powder or granular material, that is suitable for sublingual delivery.

16. The pharmaceutical dosage form of claim 15 wherein about 90% or more of the active ingredient is released from the core tablet within about 15 minutes.

17. The dosage form of claim 16 wherein the active ingredient is tizanidine and wherein about 85% or more of the tizanidine from the core tablet is released within about 15 minutes.

18. The tizanidine dosage form of claim 17 wherein the core tablet further contains crospovidone, sodium saccharine, microcrystalline cellulose and menthol.

19. The tizanidine dosage form of claim 17 wherein the annular body contains microcrystalline cellulose, sodium saccharin and crospovidone.

20. The dosage form of claim 16 wherein the rate of release is measured in a United States Pharmacopeia standard apparatus II dissolution system at 37° C. with stirring at 50 revolutions per minute.

21. A pharmaceutical dosage form for oral administration to a patient comprising a core tablet containing an active ingredient sheathed in an annular body of compressed powder or granular material, that is suitable for dissolution of the active ingredient within the oral cavity within about 5 minutes or less.

22. The dosage form of claim 21 wherein the rate of release is measured in a United States Pharmacopeia standard apparatus II dissolution system at 37° C. with stirring at 50 revolutions per minute.

23. A method of independently controlling the rate of release of coactive ingredients in a single dosage form comprising formulating one active ingredient in the core tablet of a dosage comprising a core tablet sheathed in a compressed annular body of pharmaceutical excipients and formulating a second active ingredient in the compressed annular body.

24. A pharmaceutical dosage form for co-administration of two active pharmaceutical ingredients to a patient comprising a core tablet containing a first active pharmaceutical ingredient sheathed in a annular body of compressed powder or granular material and containing a second active pharmaceutical ingredient.

25. The pharmaceutical dosage form of claim 24 wherein the first active pharmaceutical ingredient is carbidopa and the second active pharmaceutical ingredient is levodopa.

26. The pharmaceutical dosage form of claim 25 that releases levodopa from the annular body at a rate in the range of from 3% per hour to 30% per hour over a period of three hours or more.

27. The pharmaceutical dosage form of claim 26 that releases levodopa from the annular body at a rate in the range of from 6% per hour to 30% per hour over a period of three hours or more.

28. The pharmaceutical dosage form of claim 26 wherein the period of three hours or more begins from between one and two hours after contacting the dosage form with the water, the period being preceded by an initial more rapid release of carbidopa.

29. The pharmaceutical dosage form of claim 25 wherein carbidopa is completely released within about three hours after the dosage form contacts water.

30. The pharmaceutical dosage form of claim 29 wherein the carbidopa is completely release within about one hour after the dosage form contacts water.

31. The pharmaceutical dosage form of claim 26 where the rate of release is measured in 0.1 N HCl at 37° C. in a United States Pharmacopeia Apparatus II dissolution tester with stirring at 50 revolutions per minute.

32. The pharmaceutical dosage form of claim 24 wherein the core tablet further contains xylitol, crospovidone, microcrystalline cellulose and lactose.

33. The pharmaceutical dosage form of claim 24 wherein the annular body further contains ethylcellulose, powdered cellulose and lactose.

34. A pharmaceutical dosage form for oral administration to a patient comprising a core tablet containing an active ingredient sheathed in an annular body of compressed powder or granular material, that is suitable for taste masking the active ingredient.

35. The dosage form of claim 34, wherein the active ingredient is sumatriptan succinate.

36. The dosage form of claim 35 wherein the core tablet that releases about 80% sumatriptan succinate from the core tablet in about thirty minutes or less.

37. The sumatriptan succinate solid dosage form of claim 35 wherein the core tablet further contains microcrystalline cellulose, lactose and croscarmellose sodium.

38. The sumatriptan succinate solid dosage form of claim 35 wherein the annular body contains sucrose, microcrystalline cellulose and menthol.

39. The sumatriptan succinate solid dosage form of claim 35 that masks the taste of sumatriptan succinate when the dosage form is held in the mouth.
